(19) United States
(12) Plant Patent Application Publication
(10) Pub. No.: US 2002/0184682 P1
(43) Pub. Date: Dec. 5, 2002

(54) Chrysanthemum plant named 'Gedi One Ces'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,902

(22) Filed: Jun. 4, 2001

(51) Int. Cl. A01H 5/00

(52) U.S. Cl. PLT/289

(57) ABSTRACT

A new and distinct Chrysanthemum plant cultivar characterized by large decorative type inflorescences, consistent flowering response to short days, blooming consistently after 49 days of short day length, free branching habit, and bright yellow ray florets, which resist fading with age.

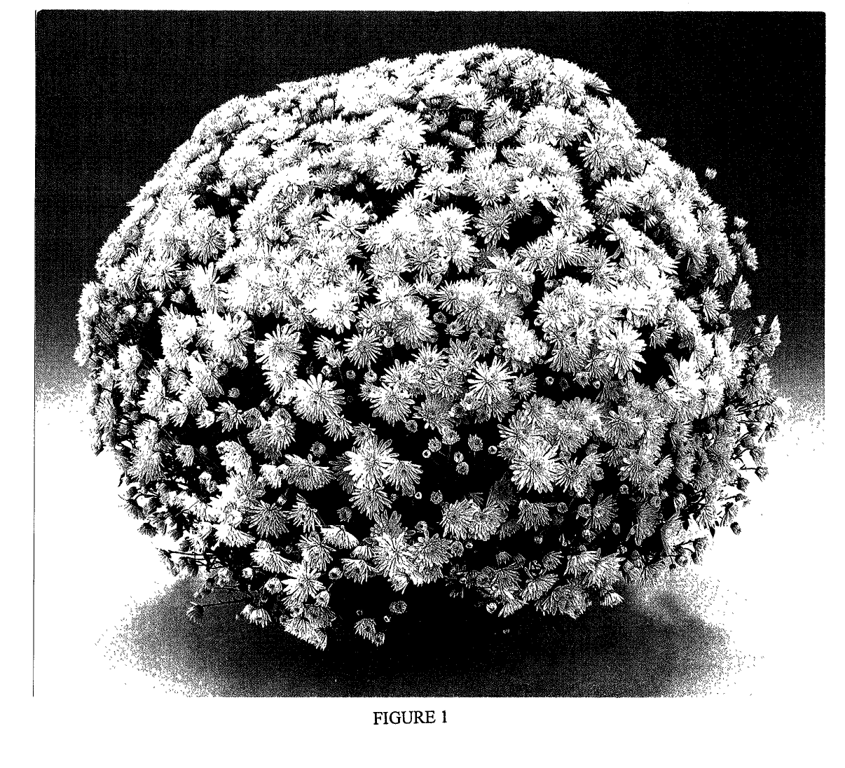

### BACKGROUND OF THE INVENTION

The present invention relates to a new and distinct cultivar of Chrysanthemum plant, botanically known as Dendranthema morifolium and hereinafter referred to by the cultivar name ‘GEDI ONE CES.’

The new cultivar is a product of hybridization of the female parent ‘Yellow Urano’ (U.S. Plant patent Application Ser. No. 09/550,153) and the male parent ‘Dark Veria’ (U.S. Plant Pat. No. 10,215). The new cultivar was discovered and selected by the inventor in October 1997.

Asexual reproduction of the new cultivar by apical tip cutting and meristem tissue culture was performed in Oxnard, CA and has shown that the unique features of this new Chrysanthemum cultivar are stable and reproduced true to type on successive generations.

### SUMMARY OF THE INVENTION

The cultivar ‘GEDI ONE CES’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE CES.’ These characteristics in combination distinguish ‘GEDI ONE CES’ as a new and distinct Chrysanthemum cultivar:

1. Large decorative type inflorescence;

2. Consistent flowering response to short days, blooming consistently after 49 days of short day length;

3. Free branching habit; and

4. Bright yellow ray florets, resisting fading with age.

Plants of the new cultivar are similar to plants of the parent cultivars, ‘Yellow Urano’ and ‘Dark Veria’ in most horticultural characteristics, however plants of the new cultivar differed from plants of the parent cultivars in several characteristics. In comparison the new cultivar differed from the parent in the following characteristics:

1. The new cultivar grows taller and wider than cultivar ‘Yellow Urano;’

2. The new cultivar has many more ray florets than the cultivar ‘Yellow Urano;’

3. The new cultivar naturally blooms two weeks later than cultivar ‘Yellow Urano;’

4. The new cultivar has individual blooms resisting fading 7 days longer than cultivar ‘Yellow Urano;’

5. The new cultivar has a wider diameter flower than cultivar ‘Dark Veria;’

6. The new cultivar has many more ray florets than the cultivar ‘Dark Veria;’

7. The new cultivar grows taller and wider than the cultivar ‘Dark Veria;’ and

8. The new cultivar naturally blooms 2 weeks earlier than the cultivar ‘Dark Veria.’

### BRIEF DESCRIPTION OF THE PHOTOGRAPHS

The photographs shows a plant of the new cultivar which was grown in a 2-gallon container. A single cutting was grown in the container, and the plant was grown in outdoor field conditions.

### DETAILED BOTANICAL DESCRIPTION

In the following description, color references are made to the Royal Horticultural Society Colour Chart except where general terms of ordinary dictionary significance are used. The following observations and measurements describe plants grown in a commercial field in Oxnard, Calif. in the month of September 2000. The growing conditions range from 60° to 65° F. at night to 70° to 85° F. during the day. Measurements and numerical values represent averages of typical flowering types. Botanical classification: Dendranthema morifolium cultivar ‘GEDI ONE CES’ Commercial classification: Garden-type Chrysanthemum Parentage: Hybridization of female parent ‘Yellow Urano’ and male parent ‘Dark Veria’

PROPAGATION

Time to Rooting.—7 to 14 days at approximately 21° C. Rooting habit: Fine, fibrous

PLANT

Growth Habit.—Mounding herbaceous perennial

Height.—Approximately 25 cm

Growth rate.—Moderate

Branching Characteristics.—Free Branching

FOLIAGE

Leaf.—Arrangement: Alternate single Average Length: Approximately 6.8 cm Average Width: Approximately 3.2 cm Shape of blade: Ovate Apex: Acuminate Base: Attenuate Attachment: Stalked Margin: Serrate Texture of top surface: Lightly pubescent Texture of bottom surface: Lightly pubescent Color: youngfoliage upper side: Near 138A youngfoliage under side: Near 138B mature foliage upper side: Near 137A mature foliage under side: Near 137A venation upper side: Near 138C venation underside: Near 138C

Petiole.—Average Length: Approximately 1.5 cm

BLOOM

Inflorescence.—Flowering habit: Induced by darkness period greater than 13.5 hours, approximately 49 days of appropriate day length required to induce and develop blooms Inflorescenceform: Decorative Natural flowering season. Approximately the last week in August and first week in September Number of inflorescences per stem: Approximately 15 Inflorescence diameter: Approximately 6 cm Inflorescence depth: 1.7 cm Inflorescence longevity on plant: Approximately 26-29 days Persistence: Persistent Ray Florets: Appearance: Matte Texture: Smooth Average Number perflower: 200 Shape: Oblanceolate Margin: Entire Apex: Obtuse Length: Approximately 3.4 cm Width: Approximately 0.8 cm Color:

upper surface atfirst opening: Near 16A upper surface at maturity: Near 17C upper surface at fading: Near 16A under surface at first opening: Near 15C under surface at maturity: Near 16A under surface at fading: Near 16B

Disc florets.—Appearance: Shiny Texture: Smooth Average number per flower: Approximately 60 Shape: Cylindric Apex: Obtuse Average Length: Approximately 1 cm Average Width: Approximately 0.2 cm Color: at first opening: Near 13C at maturity: Near 12B at fading: Near 15C

Peduncle.—Length: at terminal end (shortest): Approximately 4 cm at lateral end (longest): Approximately 9 cm Strength: Moderate Color: Near 137C Habit: Erect

Inflorescence bud.—Length: Approximately 1 cm Diameter: Approximately 1 cm Form. Globular Color: Near 15A

Sepals.—Appearance: Matte Texture: Lightly pubescent Number: Approximately 30 to 40 Shape: Ovate Margin: Entire Apex: Acute Color: upper si Near 137A under side: Near 137A

REPRODUCTIVE ORGANS

Ray Florets.—Pistillate: Number ofpistils per flower: 1 Stigma shape: 2 branched Stigma color: Near 16D Style color: Near 16D Style length: 0.5 cm

Disc Florets.—Pistillate: Number of pistils per flower: 1 Stigma shape: Cylindric Style length. 0.5 cm Staminate: Number of stamens per flower: Approximately 5 Anther shape: Tubular Anther color: Near 16A Pollen color: Near 17C

What is claimed is:

1. A new and distinct cultivar of Chrysanthemum plant named ‘GEDI ONE CES’ as herein illustrated and described.
